(19) United States
(12) Patent Application Publication
(10) Pub. No.: US 2004/0033941 A1
(43) Pub. Date: Feb. 19, 2004

(54) Immunosuppression using piceatannol and a calcineurin inhibitor

(76) Inventors: Majed M. Hamawy, Madison, WI (US); Stuart J. Knechtle, Fitchburg, WI (US)

Correspondence Address: QUARLES & BRADY LLP, 411 E. WISCONSIN AVENUE, SUITE 2040, MILWAUKEE, WI, 53202-4497, US

(21) Appl. No.: 10/363,494

(22) PCT Filed: Jul. 25, 2001
(86) PCT No.: PCT/US01/23564

(51) Int. Cl. A61K 38/13; A61K 31/704; A61K 31/4745

(52) U.S. Cl. 514/11; 514/25; 514/291

(57) ABSTRACT

Disclosed herein are methods and drugs for suppressing acute/chronic rejection responses of a transplant recipient. Piceatannol and calcineurin inhibitors such as cyclosporin A and FK506 are administered in a combined protocol shortly before, and/or after, the transplant. They can be injected in an organic solvent, or other carrier.

### CROSS-REFERENCE TO RELATED APPLICATIONS

This is a continuation-in-part provisional of U.S. provisional application No. 60/228,551, filed Aug. 28, 2000.

### STATEMENT REGARDING FEDERALLY SPONSORED RESEARCH OR DEVELOPMENT

This invention was made with United States government support awarded by NIH: 5R01AI40597-02. The United States has certain rights in this invention.

### BACKGROUND OF THE INVENTION

The present invention relates to methods for suppressing acute/chronic rejection responses of a transplant recipient. More particularly, it involves the use of piceatannol (3,4,3′,5′-tetrahydroxy-trans-stilbene) in combination with a calcineurin inhibitor such as cyclosporin A or FK506 to suppress adverse transplant rejection symptoms.

Transplantation is an important therapeutic option for patients, particularly those with end-stage organ diseases. Immunosuppressive strategies have increased the rate of transplant success. However, many prior art immunosuppressants have undesirable side effects, are not well tolerated over long periods by certain recipients, have too high a cost, and/or rely on a suppression mechanism that causes some damage to be done to the transplanted organ/cell before the suppression takes effect.

One class of the more widely used immunosuppressive agents is calcineurin inhibitors, particularly cyclosporin A and FK506. Cyclosporin A inhibits transcription of certain genes important to the immune response, such as IL-2. Cyclosporin A binds with cyclophilin, and the resulting complex binds to and inhibits the enzymatic activity of the calcium/calmodulin-activated protein phosphatase known as calcineurin.

Since calcium/calmodulin-activated calcineurin function is required to activate a cytoplasmic component of the transcription factor NFAT, cyclosporin A blocks NFAT activation and the transcription of IL-2. Cyclosporin A thus blocks the IL-2-dependent growth and differentiation of T cells. Unfortunately, prolonged use of levels of cyclosporin A that are sufficient for immunosuppression (typically 5-20 mg/kg of body weight per day) has been shown in some cases to cause adverse side effects (e.g. kidney damage).

The fungal metabolite FK506 is commercially available from Fujisawa USA, Inc. (Deerfield, Ill.). The complex of FK506 and its binding protein (called FKBP) bind calcineurin and thereby also inhibit its action. Thus, it is also a calcineurin inhibitor. Problems associated with use of prolonged high levels of FK506 for immunosuppression include nephrotoxicity and neurotoxicity. See generally P. Tsuchida et al., 95 Blood 2733-41 (2000)(calcineurin inhibitors cyclosporin A and FK506 have therapeutic application).

In unrelated work, piceatannol was isolated from plants. It is now commercially available and can readily be synthesized-by procedures such as that described in R. Bajaj et al., 18 Rev. Latinoamer. Quim. 80-83 (1987).

In connection with investigating anti-cancer properties of piceatannol, it was learned that piceatannol acts as an inhibitor of protein-tyrosine kinases. See generally N. Ferrigni et al., 47 J. Nat. Prod. 347-352 (1984); M. Gill et al., 50 J. Nat. Prod. 36-40 (1987); and R. Geahlen et al., 165 Biochem. Biophys. Res. Com. 241-245 (1989). The disclosure of these publications, and all other publications referred to herein, are incorporated by reference as if fully set forth herein.

It was later learned (in connection with mast cell/allergy development studies) that piceatannol was a very effective inhibitor of Syk kinase. J. Oliver et al., 269 J. Biol. Chem. 29697-29703 (1994). We then tried using piceatannol as an immunosuppressive. While it did have desirable immunosuppressive properties, in order to achieve desirable inhibition by itself levels of piceatannol needed to be used which were not well tolerated (e.g. 75 mg/kg of body weight per day).

Thus, the need still exists to develop improved immunosuppressive therapies for use with transplant recipients.

### BRIEF SUMMARY OF THE INVENTION

In one aspect the invention provides a method for inhibiting a rejection response of a primate (e.g. a human) transplant recipient who has received a transplanted cell from a donor primate (e.g. a donor human provided an organ). One administers to the recipient both piceatannol and a calcineurin inhibitor such that a rejection response is inhibited. Preferably the calcineurin inhibitor is selected from the group consisting of cyclosporin A and FK506.

By inhibiting “a rejection response” we mean inhibiting an adverse acute or chronic immunological response that if not suppressed would ultimately lead to transplant rejection. Among other things, this could involve tubulitis, intimal arteritis, cellular infiltrates, glomerulosclerosis, interstitial fibrosis, vascular obliterative changes, coronary arteriosclerosis, bronchiolitis obliterans, and biliary occlusion.

In one form the administration is by injecting the piceatannol and a calcineurin inhibitor as a mixture into the primate, such as by intravenous or intramuscular injection. A preferred protocol is for between 5 mg and 40 mg (preferably 30 mg) of piceatannol per kilogram of the recipient's body weight, and between 1 mg and 3 mg of cyclosporin A (preferably 2 mg) per kilogram of the recipient's body weight. This is to be administered to the recipient on at least one day (and preferably for three days before and for at least seven days after transplant).

Based on toxicity concerns, it is desirable that the dosage of piceatannol on any given day or on average not exceed 50 mg/kg of body weight. Further, given concerns relating to other adverse side effects it is desirable that the dosage of cyclosporin A on any given day or on average not exceed 4 mg/kg of body weight. For FK506, the preferred dosage is expected to be about 0.5 mg/kg of body weight per day.

The piceatannol and calcineurin inhibitor are preferably administered to the recipient at least to some extent prior to the recipient receiving the transplanted cell (e.g. beginning more than two days prior to the recipient receiving the transplanted cell).

In another preferred form, treatment can be continued between day 8 and day 60 after transplant, albeit with the piceatannol levels reduced to about 10 mg/kg. Thereafter, the treatment can be discontinued as tolerance has been achieved.

Thus, in another aspect, the invention provides a method for causing a primate transplant recipient to develop tolerance to a transplanted cell from a donor primate. One administers to the recipient both piceatannol and a calcineurin inhibitor such that such tolerance is achieved. For purposes of this application, tolerance is defined as the recipient being able to prevent a rejection response in the absence of ongoing therapy over a period of at least thirty days.

The above methods are particularly suitable for use in connection with the transplantation of organs (especially heart, lung, liver, and kidney). It may also be part of a skin graft or bone marrow transplant.

In another form the invention provides an immunosuppressive drug which is a mixture of piceatannol and a calcineurin inhibitor selected from the group consisting of cyclosporin A and FK506.

Piceatannol is particularly effective in inhibiting the kinase activity of two kinases (Syk and Zap). These are found in T cells and B cells in concentrations that are considerably higher than in other cells. These protein tyrosine kinases (“PTKs”) are involved in an early stage of the immunological cascade that follows a T cell encountering a foreign “intruder”.

In contrast, the calcineurin inhibitors inhibit the activation of B and T cells much later in the biochemical cascade. It has been surprisingly discovered that due to this two-stage simultaneous attack rejection inhibition can be achieved at surprisingly low levels of piceatannol and calcineurin inhibitor.

The advantages of the present invention include providing methods of the above kind:

(a) that inhibit a rejection response of a transplant recipient;

(b) that permit such a recipient to develop tolerance to a transplanted organ;

(c) which have few side effects; and

(d) which have relatively low cost.

These and still other advantages of the present invention will be apparent from the description which follows. The following description is merely of the preferred embodiments. The claims should be looked to in order to understand the full scope of the invention.

### BRIEF DESCRIPTION OF THE DRAWINGS

FIG. 1 is a graph showing the results of various immunosuppressive treatments used on animals who had received a kidney transplant.

### DETAILED DESCRIPTION OF THE INVENTION

### Overall Approach

T and B lymphocytes are central to transplant rejection. They have surface antigen receptors by which they recognize an “intruder” such as a transplanted organ or cell. The binding of T and B cells to the transplanted organ or cell activates the lymphocytes and leads to the release of various cytokines and/or antibodies that ultimately can cause rejection.

Whereas T cells are involved in regulating the response of immune cells and in directly attacking and killing allocells, B cells are devoted primarily to the production of anti-graft antibodies. Hence, inhibiting both T and B cell functions is desirable for immunosuppression.

A main route for T and B cell activation in vivo is the aggregation of their surface antigen receptors (AgR). The cross linking of AgR by antigens initiates a cascade of intracellular reactions including protein tyrosine phosphorylation, phospholipid hydrolysis, protein kinase C activation and calcium influx.

Our studies have confirmed that piceatannol inhibits T and B cell activation both in vitro and in vivo. Further, we have found that in vivo use of piceatannol prolongs transplanted organ survival, apparently by inhibiting protein tyrosine phosphorylation by suppressing Syk and Zap kinases, albeit at levels in the 75 mg/kg/day range some toxicity was noted.

In this regard, Zap and Syk are known to be important for T and B cell function. See generally M. Elder et al., 264 Science 1596-1599 (1994); I. Negishi et al., 376 Nature 435-438 (1995); A. Cheng et al., 378 Nature 303-306 (1995); and A. Chan et al., 71 Cell 649-662 (1992); R. Soede et al., 142 J. Cell. Biol. 1371-79 (1998); M. Tsuchida et al., 29 Eur. J. Immunol. 2354-59 (1999); M. Tsuchida et al., 284 J. Biol. Chem. 6735-40 (1999).

In T and B cells the activation of PTKs is the earliest known response after AgR aggregation. Protein tyrosine phosphorylation can be detected within a few seconds of receptor cross linking. An initial step in the AgR signaling cascade is the tyrosine phosphorylation of the cytoplasmic domains of the receptor's subunits, a process that is mediated by the receptor-associated PTKs. Once phosphorylated, the receptors act as a docking site for the PTK Zap-70 in T cells, and the PTK Syk in B cells.

The binding of Zap-70 and Syk to their sites on the receptors activates these PTKs and leads to the tyrosine phosphorylation of a variety of downstream molecules including phospholipase C gamma, an enzyme that regulates phosphatidylinositol turnover, and in turn, intracellular calcium levels. Piceatannol also has the advantage of preferentially inhibiting tyrosine kinases. As such, it is less likely to affect cells that do not contain Syk or Zap.

As noted previously, calcineurin inhibitors such as cyclosporin A and FK506, have also previously been used to prevent rejection in transplant patients. However, in cases of prolonged use unacceptable side effects can occur when the dosage is sufficiently high to be effective.

We reduce the amount of calcineurin inhibitor used (to less than half of conventional levels). We also reduce the amount of piceatannol from levels noted by us to cause toxicity. By combining these two inhibitors at reduced levels we are able to achieve synergistic inhibition.

### General Materials and Methods

In vivo studies were conducted using piceatannol obtained from Peninsula Lab. Inc. (Belmont, Calif.), and cyclosporin A was obtained from Sandoz (East Hanover, N.J.). We examined the ability of piceatannol/calcineurin mixtures to prolong graft survival. Donor organs from ACI rats were transplanted into Lewis rats. These rats are a standard model for assessing likely mammalian (including primate) response to immunosuppressive therapy.

Prior to transplantation, piceatannol (30 mg/kg) was administered intravenously for three days into one selection of Lewis rats via a catheter placed in the right jugular vein. Another selection of such rats received only cyclosporin A (2 mg/kg/day) intramuscularly during this period using a 27G fine needle. A third group received cyclosporin A (2 mg/kg/day) in combination with piceatannol (30 mg/kg/day) intramuscularly. A fourth group were controls that had not received any immunosuppressive drug.

In these tests, the piceatannol was dissolved in dimethyl sulfoxide (DMSO) (Sigma Chemical, St. Louis, Mo.), and the cyclosporin A was dissolved in polyoxyl 35 castor oil with 32.9% ethanol. This carrier is also a suitable carrier for FK506.

### Heart Experiments

At day 3, hearts from ACI rats were heterotopically transplanted into the abdomen of the Lewis rats. The donor heart was excised after ligating the inferior and superior vena cava and the pulmonary veins. The heart was then anastomosed to the abdominal aorta and inferior vena cava of the recipient rat through a laparotomy incision. Following transplantation, fascia and muscle were closed with absorbable suture and skin was closed with nylon.

After surgery animals were placed on a warm pad and observed until they awakened. The animals were then placed in cages in a warm environment with wood chip bedding, and provided rodent chow. To relieve pain, Buprenorphine was also given to the animal. Toxicity in animals was monitored by measuring the blood levels of creatinine and ALT, indicators of kidney and liver function, respectively. Drug administration then continued for an additional seven days after the transplant surgery (and was then discontinued).

In the case of heart transplants, rejection was considered complete when no heart beat was palpable with respect to the transplanted organ. This was confirmed by laparotomy and direct inspection.

All animals receiving heart transplants that received neither piceatannol nor cyclosporin A (the controls) rejected the graft by the end of day 7. As can be seen from the following tables, while piceatannol alone, or cyclosporin A alone, at the specified dosages had only minimal prolongation effect, the combination of the two radically increased survival.

| Group # | Drugs | Graft Survival (days) |
|---|---|---|
| 1 | none | 6, 6, 7, 7, 6, 6, 6, 6, 6 |
| 2 | 30 mg/kg piceatannol | 6, 7, 8, 5, 7, 10 |
| 3 | 2 mg/kg cyclosporin A | 8, 10, 10, 11, 11, 11, 12 |
| 4 | 2 mg/kg cyclosporin A + | 10, 16, 24, 12, 6, 8, 14, |
| | 30 mg/kg piceatannol | 16, 18, 15, 18 |

### Kidney Experiments

In other experiments, kidneys from ACI rats were transplanted into Lewis rats. Drug administration continued for an additional seven days after transplant at the same levels. From day 8 through day 60 post-transplant administration continued, albeit with piceatannol reduced to 10 mg/kg/day. Thereafter, drug administration ceased.

Animals were anesthetized. The lower abdomen of the donor was shaved and surgically prepared and a long midline incision was made in the abdominal wall. The left kidney and left ureter, aorta, and vena cava were dissected, and suprarenal and gonadal veins ligated and cut. After ligating the suprarenal aorta and vena cava, a hole made in distal aorta and left kidney was flushed in situ with 3 ml of heparinized cold Euro-Collins' solution. The left kidney and left ureter were removed with an aortic cuff and vena cava patched and kept on ice.

After the lower abdomen of the recipient was shaved and surgically prepared, the recipient animal underwent a left nephrectomy via a long midline incision under inhalation anesthesia, and abdominal aoerta and vena cava were prepared for anastomosis. The donor renal artery was anastomosed end to side to the recipient abdominal aorta via the aortic cuff with 8-0 suture. The donor renal vein was anastomosed end to side to the recipient infrarenal IVC via a vena cava patch with 10-0 suture. The donor ureter was sutured to the recipient ureter using 10-0 interrupted suture.

The abdominal wound was closed in two layers, one of continued 3-0 plain catgut stitches in the muscle layer and the other of continued 4-0 Dermalon or Surgilene stitches in the skin. Three days later, a long midline incision was made in the abdominal wall of the recipient rat under anesthesia. The right kidney was separated from the peri-renal fat and suprarenal gland and the kidney was removed after placing a single tie around the renal pedicle. The abdominal incision was closed as described above after the nephrectomy.

This nephrectomy was delayed until 3 days after the graft to allow time for the donor kidney to recover from any ischemic damage. The animal was allowed to recover in its cage and then re-observed an hour later for any complications.

As graphed on FIG. 1, for kidney transplantation, the animals were divided into the following groups. Group 1 (n=6) received piceatannol 30 mg/kg/day IV and CsA 2 mg/kg/day IM from day −3 to +7 post transplant. At day 8, the dose of piceatannol was reduced to 10 mg/kg/day and the combined treatment was resumed until day +60. Group 2 (n=7) received 2 mg/kg/day CsA (IM) alone from day −3 to +60. Group 3 (n=4) received piceatannol (IV) alone using the same protocol as for Group 1. Group 4 (n=2) received only the vehicle DMSO (IV).

Graph rejection was defined as either serum creatinine level over 2 mg/dl or animal death, whichever happened earlier. As shown in FIG. 1, DMSO, piceatannol, or CsA alone were not effective in prolonging allograft survival. Histological analysis of transplanted kidneys showed signs of acute rejection in all these animals (data not shown). Six out of seven animals treated with only 2 mg/kg/day of CsA rejected their grafts within 10 days of transplantation. The seventh animal in this group survived for 60 days.

In contrast all six animals treated with the combination of piceatannol and the subtherapeutic dose of CsA did not reject their allografts, as excellent kidney function was maintained for more than 60 days (FIG. 1). Although some initial weight loss (˜10% of body weight) was observed in these animals, the animals regained weight when the dose of piceatannol was reduced at day 8 to 10 mg/kg/day. At day 60, all the animals treated with the combination of piceatannol and CsA were active and did not show signs of stress or lethargy.

### Other Embodiments

While the initial experiments were made on rat recipients of heart and kidney transplants, the present invention is suitable for use on a wide range of mammals, including without limitation primates such as humans. The preferred method of administration for humans is via intraperitoneal or subcutaneous injection. However, intravenous and other forms of administration are likely to be suitable as well.

As piceatannol is hydrophobic, and as injection is a possible delivery technique, the piceatannol can be dissolved in any organic solvent carrier which the body accepts well (e.g. ethanol), and/or in solvent/saline mixtures. Other liquid carriers that are compatible with the host are also possible. The piceatannol can be delivered in one injection, with the calcineurin inhibitor being delivered in another injection with carriers conventional for their use alone (e.g. for cyclosporin A polyoxyl 35 castor oil—Cremophor EL, polyetholxylated castor oil—with 32.9% ethanol). Alternatively, both can be mixed together in a suitable carrier.

Our technique is also suitable for use with a wide range of other transplantable organs (particularly lung, pancreas and liver). It also should be useful in connection with transplantation of tissues and cells (e.g. skin grafts, bone marrow, islets transplants).

The administered dosage in each case will need to be optimized based on the degree of rejection the host is likely to experience with absence of the drug (e.g. how close a match the donor organ is), the size of the recipient, what other immunosuppressive drugs are simultaneously being used, the medical condition of the recipient, whether the drug is delivered pre- or post-transplant, and whether the dosage is being repeated. However, it is generally desirable to have a dosage of at least 25 mg/kg of piceatannol for at least 3 days before and at least 7 days after the transplant.

The piceatannol dissolved in a suitable organic solvent should typically be stored at −20° C. until use, then thawed to room temperature, diluted in a suitable aqueous saline solution if desired, and injected. The condition of the patient should be monitored in the usual manner.

For a calcineurin inhibitor such as cyclosporin A, it is generally desirable to have a dosage of at least 1 mg/kg/day for at least 3 days before and at least 7 days after the transplant.

Thus, the invention is not to be limited to the specific embodiments described above. Rather, it should be provided its full scope as described in the following claims.

### INDUSTRIAL APPLICABILITY

The invention provides methods and drugs to suppress acute/chronic rejection responses of transplant recipients.

We claim:

1. A method for inhibiting a rejection response of a primate transplant recipient who has received a transplanted cell from a donor primate, comprising:
administering to the recipient both piceatannol and a calcineurin inhibitor such that the rejection response is inhibited.

2. The method of claim 1, wherein the calcineurin inhibitor is selected from the group consisting of cyclosporin A and FK506.

3. The method of claim 1, wherein the administration is by injecting the piceatannol and a calcineurin inhibitor as a mixture into the primate.

4. The method of claim 3, wherein the injection is selected from the group consisting of intravenous injection and intramuscular injection.

5. The method of claim 1, wherein the donor primate and recipient primate are two different humans.

6. The method of claim 1, wherein between 5 mg and 40 mg of piceatannol, and between 0.01 mg and 3 mg of the calcineurin inhibitor, per kilogram of the recipient's body weight, are administered to the recipient on at 5 least one day.

7. The method of claim 1, wherein the piceatannol and calcineurin inhibitor are administered to the recipient at least in part prior to the recipient receiving the transplanted cell.

8. The method of claim 7, wherein the piceatannol and calcineurin inhibitor are administered to the recipient at least in part more than two days prior to the recipient receiving the transplanted cell.

9. The method of claim 1, wherein the transplanted cell is part of an organ from a donor and the organ has been transplanted from the donor to the recipient.

10. The method of claim 9, wherein the organ is selected from the group consisting of heart and kidney.

11. A method for causing a primate transplant recipient to develop tolerance to a transplanted cell from a donor primate, comprising:
administering to the recipient both piceatannol and a calcineurin inhibitor such that such tolerance is achieved.

12. The method of claim 11, wherein the tolerance is achieved within sixty days after the cell has been transplanted into the recipient.

13. The method of claim 11, wherein the calcineurin inhibitor is selected from the group consisting of cyclosporin A and FK506.

14. The method of claim 11, wherein the administration is by injecting the piceatannol and a calcineurin inhibitor as a mixture into the primate.

15. The method of claim 14, wherein the injection is selected from the group consisting of intravenous injection and intramuscular injection.

16. The method of claim 11, wherein the donor primate and recipient primate are two different humans.

17. The method of claim 11, wherein between 5 mg and 40 mg of piceatannol, and between 0.01 mg and 3 mg of the calcineurin inhibitor, per kilogram of the recipient's body weight, are administered to the 5 recipient on at least one day.

18. The method of claim 11, wherein the piceatannol and calcineurin inhibitor are administered to the recipient at least in part prior to the recipient receiving the transplanted cell.

19. The method of claim 18, wherein the piceatannol and calcineurin inhibitor are administered to the recipient at least in part more than two days prior to the recipient receiving the transplanted cell.

20. The method of claim 11, wherein the transplanted cell is part of an organ from a donor and the organ has been transplanted from the donor to the recipient.

21. The method of claim 20, wherein the organ is selected from the group consisting of heart and kidney.

22. An immunosuppressive drug comprising a mixture of piceatannol and a calcineurin inhibitor selected from the group consisting of cyclosporin A and FK506.
